(19) United States
(12) Patent Application Publication
(10) Pub. No.: US 2016/0066793 A1
(43) Pub. Date: Mar. 10, 2016

(54) OBJECT INFORMATION ACQUIRING APPARATUS

(71) Applicant: CANON KABUSHIKI KAISHA, Tokyo (JP)

(72) Inventors: Takeshi Yamamoto, Tokyo (JP); Robert A Kruger, Oriental, NC (US)

(21) Appl. No.: 14/838,913

(22) Filed: Aug. 28, 2015

Related U.S. Application Data
(60) Provisional application No. 62/046,352, filed on Sep. 5, 2014.

(51) Int. Cl. A61B 5/00

(52) U.S. Cl. CPC A61B 5/0095; A61B 5/704; A61B 5/4312; A61B 5/708; A61B 2562/14

(57) ABSTRACT

An object information acquiring apparatus including a receiver provided with: a plurality of detecting elements which receive an acoustic wave from an object and generate an electrical signal and provided with a container-shaped supporter which supports the plurality of detecting elements; and a tank which holds an acoustic matching liquid; and a flow path which connects the tank with a liquid inlet port and a liquid discharge port provided in the receiver, the object information acquiring apparatus further including: a liquid circulator which controls a flow of the acoustic matching liquid; a UV irradiator which irradiates the acoustic matching liquid with ultraviolet light onto; and a processor which uses the electrical signal to generate property information on the interior of the object.

### BACKGROUND OF THE INVENTION

1. Field of the Invention

The present invention relates to an object information acquiring apparatus.

2. Description of the Related Art

In recent years, research and development has been carried out into photoacoustic imaging apparatuses which create images of the interior of a living body by using a photoacoustic effect. A photoacoustic imaging apparatus emits pulse laser light (laser pulses) emitted at short durations into a living body, and generates an image from the ultrasound waves (photoacoustic waves) that are created by swelling of the volume of the living tissue due to the generation of heat upon absorbing the energy of the pulse laser light. Photoacoustic imaging apparatuses have been researched and developed as apparatuses for observing a human breast for the purpose of early discovery of breast cancer, for example.

One example of the specific configuration of a photoacoustic imaging apparatus is disclosed in Japanese Patent Application Laid-open No. 2012-179348. The apparatus disclosed in Japanese Patent Application Laid-open No. 2012-179348 emits light and receives acoustic waves by acoustic detecting elements, while relatively scanning an object and an acoustic array detector in which a plurality of acoustic detecting elements are arranged. The acoustic detecting elements output a signal due to receiving the acoustic waves. By using this signal to image reconstruction, it is possible to acquire image data.

An acoustic matching agent for acoustically coupling the object and the acoustic array detector is filled in between the object and the acoustic array detector. Since the object and the acoustic array detector are scanned relative to each other, the acoustic matching agent is desirably a material capable of deforming freely between the object and the acoustic array detector (typically, a liquid). Moreover, the material of the acoustic matching agent is desirably a liquid that has an acoustic impedance close to that of the object and the acoustic detecting elements, and which allows the transmission of pulsed light.

Patent Literature 1: Japanese Patent Application Laid-open No. 2012-179348

Patent Literature 2: Japanese Patent No. 4341987

### SUMMARY OF THE INVENTION

When a liquid (acoustic matching liquid) is used as an acoustic matching agent for a long period of time, there is a possibility that dust in the air may become mixed into the liquid, and that organic material such as mold or lichen may occur. Deterioration in the acoustic matching liquid of this kind risks giving rise to health problems and adverse effects on image quality. Furthermore, there is also a problem in that, if mold or lichen, etc. occurs on the surface of the holding member which holds the object and/or the acoustic array detector disclosed in Japanese Patent Application Laid-open No. 2012-179348, then cleaning work is required.

If the acoustic matching liquid is replaced frequently due to the infiltration of dust and/or the growth of organic materials, a reducing effect is obtained. However, if the acoustic matching liquid is replaced with considerable frequency, then costs increase. Furthermore, the replacement work takes time, and there is a risk of decline in the usability of the apparatus.

The present invention was devised in view of the abovementioned problem, an object thereof being to provide an object information acquiring apparatus which is capable of suppressing deterioration of an acoustic matching liquid, without replacing the acoustic matching liquid.

The present invention provides an object information acquiring apparatus, comprising:

a receiver provided with a plurality of detecting elements which receive an acoustic wave from an object and generate an electrical signal, and provided with a container-shaped supporter which supports the plurality of detecting elements;

a tank which holds an acoustic matching liquid;

a liquid circulator which is provided with a flow path connecting the tank with a liquid inlet port and a liquid discharge port provided in the receiver and which controls a flow of the acoustic matching liquid;

a UV irradiator which irradiates the acoustic matching liquid with ultraviolet light; and

a processor which uses the electrical signal to generate property information on the interior of the object.

According to the present invention, it is possible to provide an object information acquiring apparatus capable of suppressing deterioration of an acoustic matching liquid, without replacing the acoustic matching liquid.

Further features of the present invention will become apparent from the following description of exemplary embodiments with reference to the attached drawings.

### BRIEF DESCRIPTION OF THE DRAWINGS

FIG. 1 is a block diagram of an object information acquisition apparatus;

FIG. 2 is a conceptual diagram of an apparatus according to the present invention;

FIG. 3 is a diagram showing a liquid circulator of an apparatus according to the present invention;

FIG. 4 is a diagram showing a configuration of a UV irradiator;

FIG. 5 is a diagram illustrating the configuration of a second embodiment;

FIG. 6 is a diagram illustrating the configuration of a third embodiment; and

FIG. 7 is a diagram illustrating the configuration of a fourth embodiment.

### DESCRIPTION OF THE EMBODIMENTS

Below, preferred embodiments of the present invention are described with reference to the drawings. The dimensions, materials, shapes and relative positions, and the like, of the constituent parts described in these embodiments should be changed appropriately depending on the composition and various conditions of the apparatus to which the invention is applied, and it is not intended to limit the scope of the invention to the description of the embodiments given below.

The present invention relates to technology for detecting an acoustic wave propagated from an object, and generating and acquiring property information of the inside of the object. Therefore, the present invention may be understood as an object information acquiring apparatus or control method for same, or an object information acquiring method or a signal processing method. The present invention may also be understood as a program which causes an information processing apparatus provided with a CPU and/or other hardware resources to execute these methods, or a storage medium on which this program is stored. The present invention may also be understood as an acoustic wave measuring apparatus and/or a control method for same.

The present invention can be applied to object information acquiring apparatuses using photoacoustic tomography, in which light (electromagnetic wave) is emitted onto an object, and an acoustic wave generated and propagated at a specific position inside the object or on the surface of the object, due to a photoacoustic effect, is received (detected). An apparatus of this kind is also called a photoacoustic image-forming apparatus, or photoacoustic imaging apparatus, or simply a photoacoustic apparatus, since property information for the interior of an object is obtained in the form of image data and/or property distribution information, etc., on the basis of photoacoustic measurement.

The property information in the photoacoustic apparatus is, for instance, the distribution of a generation source of an acoustic wave which is generated by the irradiation of light, the initial sound pressure distribution inside the object, or a light energy absorption density distribution or absorption coefficient distribution derived from the initial sound pressure distribution, and the density distribution of the material constituting tissue. The density of the material is, for instance, the oxygen saturation, oxyhemoglobin density, deoxyhemoglobin density, total hemoglobin density, or the like. The total hemoglobin density is the sum of the oxygenated hemoglobin density and the deoxygenated hemoglobin density. Furthermore, the distribution of fat, collagen, water, or the like, may also be the subject of the property information. Furthermore, the property information may be determined as distribution information for each position inside the object, rather than as numerical data. In other words, the object information may be distribution information, such as the absorption coefficient distribution or oxygen saturation distribution, or the like.

The present invention can also be applied to a apparatus which uses ultrasound echo technology in which an ultrasound wave is transmitted to the object, a reflected wave (echo wave) reflected inside the object is received, and object information is acquired as image data. In the case of an apparatus using ultrasound echo technology, the acquired object information is information that reflects differences in the acoustic impedance of the tissue inside the object.

The acoustic wave referred to in the present invention is typically an ultrasound wave, and includes elastic waves called sound waves or acoustic waves. An acoustic wave generated by the photoacoustic effect is called a photoacoustic wave or optical ultrasound wave. An electrical signal (reception signal) converted from an acoustic wave by a probe may also be called an acoustic signal, and an acoustic signal originating from a photoacoustic wave is called a “photoacoustic signal” in particular.

A human breast is envisaged as the object in the present invention. However, the object is not limited to this and investigation of another segment of a living body, or of non-living material, is also possible.

### First Embodiment

(Overall Description of Apparatus)

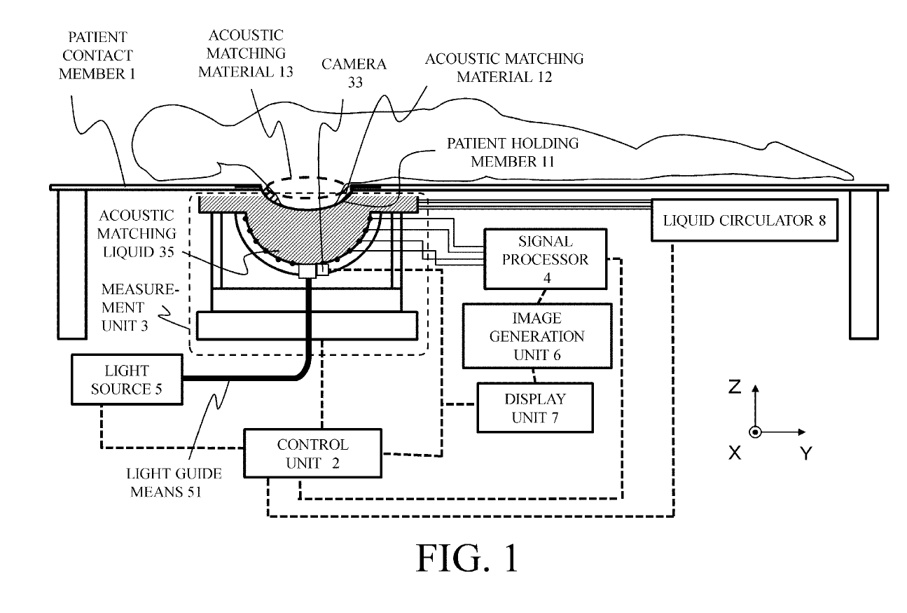

FIG. 1 is a block diagram of an object information acquiring apparatus according to the present invention.

A patient adopts a prone lying posture above an object contact member 1, and the patient's breast hangs down from a breast insertion opening 13, which is an opening in the object contact member 1, and makes contact with the holding member 11. The object contact member 1 is a member which has the strength to support the patient. When the various constituent elements of the object information acquiring apparatus according to the present invention are accommodated in a frame, the object contact member 1 may be formed by providing an opening in the upper surface of the frame.

As described in detail below, a measurement unit 3 irradiates the breast which is held by the holding member 11 with light energy from a light source 5. In so doing, a photoacoustic wave is generated from the surface of the breast and/or a light absorbing body inside the breast. A matching liquid 35 is filled in between the measurement unit 3 and the holding member 11, and an acoustic wave generated from the breast is received by a reception element of the measurement unit 3, via the matching liquid 35, and is output as a reception signal (electrical signal). The intensity of the emitted light, the timing of light irradiation and reception of the acoustic wave, and the scanning of the detector, which is described below, etc. are controlled by a controller 2. The electrical signal originating from the received acoustic wave is converted from analog into digital data (photoacoustic data) by a signal processor 4. An image generation unit 6 generates a two-dimensional or three-dimensional photoacoustic image using the photoacoustic data. The photoacoustic image thus generated, and the image acquired by a camera 33 are displayed on a display unit 7.

(Holding Member)

The irradiation light emitted onto the breast decays upon travelling through the breast, and therefore the intensity of the irradiation light is lower in the deeper segments of the living body. Therefore, it is possible to increase the intensity of the irradiation light that reaches the interior of the object, by pressing the breast hanging from the holding member 11 so as to make the breast thinner and broader. Furthermore, by pressing the breast against the holding member 11 during imaging, it is possible to reduce the effects of body movements, and therefore to suppress blurring of the image obtained by image reconstruction. The holding member 11 is a member that transmits light, and furthermore, desirably is configured by a member which is sufficiently thin to enable ultrasound waves to be transmitted therethrough easily (0.1 to 0.5 mm) and which has a strength that can withstand the body weight of the patient. The space between the holding member 11 and the breast is filled with an acoustic matching material 12, so as to permit easy transmission of ultrasound waves. Examples of the acoustic matching material 12 are water or a gel, etc. It is desirable to prepare a plurality of holding members and to exchange the holding member in accordance with the size and shape of the breast. The receiver has a container shape which can be filled with the acoustic matching liquid, and for convenience in introducing the object into the opening in the container, the holding member 11 is disposed above the receiver, as shown in the drawings.

(Light Source)

When the object is a living body, light of a particular wavelength that is absorbed by specific components among the components constituting the breast is irradiated from the light source 5. When this light is absorbed by the object, a photoacoustic wave is generated from the object. The light source may be provided in an integrated fashion with the photoacoustic apparatus of the present embodiment, or may be provided as a separate body from the light source. The light source is desirably a pulsed light source which is capable of generating, as irradiation light, light pulses of the order of several nanoseconds to several hundred nanoseconds. More specifically, in order to generate a photoacoustic wave efficiently, a pulse width of approximately 10 to 100 nanoseconds is used. A laser is desirable as the light source, due to having a high output, but it is also possible to use a light-emitting diode, or the like, instead of a laser. For the laser, it is possible to use lasers of various kinds, such as a solid laser, gas laser, fiber laser, dye laser, semiconductor laser, or the like. The timing, waveform, intensity, and the like, of irradiation is controlled by a light source controller, which is not illustrated. The wavelength of the light source used in the present invention desirably employs a wavelength at which the light is propagated to the interior of the breast. More specifically, the wavelength is no less than 500 nm and no more than 1200 nm.

(Measurement Unit)

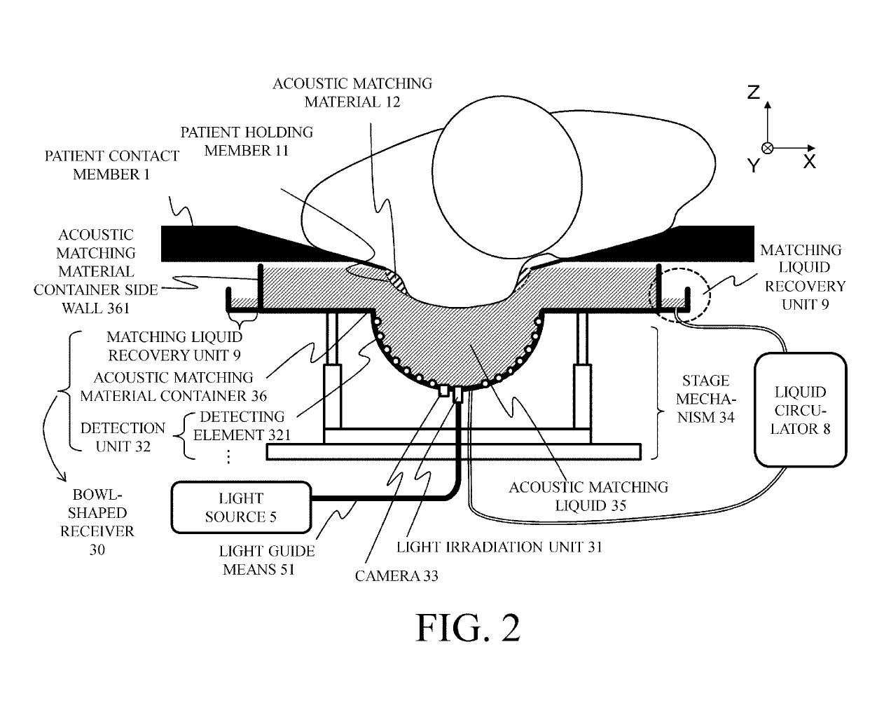

FIG. 2 is a schematic drawing of a measurement unit according to the present invention. The measurement unit has a stage mechanism 34 which enables two-dimensional movement of a bowl-shaped receiver 30, with respect to the object, in an XY plane, or three-dimensional movement of same including the Z direction. The bowl-shaped receiver 30 is configured by a detection unit 32 constituted by two or more detecting elements 321 which receive an acoustic wave generated from the breast, an acoustic matching material container 36 which can fill an acoustic matching liquid 35 in between the detection unit 32 and the object holding member 11, and a matching liquid recovery unit 9. A camera 33 for observing the held state of the breast, and a light irradiation unit 31 which emits laser light from the light source 5 guided using light guide means 51, onto the breast, are arranged at a position directly opposing the object (breast) in the acoustic matching material container 36.

By means of the configuration described above, the photoacoustic wave generated by thermal expansion when laser light is emitted onto the breast is received by the detection unit 32, which is constituted by a plurality of detecting elements 321. Water, or the like, which has an acoustic impedance closer to that of the human body, compared to air, is used for the acoustic matching liquid 35, and is supplied to the acoustic matching material container 36 by a liquid circulator 8, which is described hereinafter.

(Detecting Elements)

The detecting elements 321 detect an acoustic wave and convert same into an electrical signal, which is an analog signal. Any kind of element may be used, provided that the element is capable of detecting an acoustic wave, such as a converting element using a piezoelectric effect, a converting element using resonance of light, or a converting element using change in capacitance, etc. In the present embodiment, a plurality of detecting elements 321 are arranged, and each of the detecting elements is disposed in such a manner that their directions of greatest reception sensitivity are mutually different. By using a multi-dimensional arrangement of elements in this way, it is possible to detect an acoustic wave in a plurality of locations, simultaneously, and hence the detection time can be shortened. Furthermore, the SN ratio is improved.

(Detection Unit/Acoustic Matching Material Container)

The detection unit 32 is desirably configured in such a manner that a plurality of detecting elements 321 are arranged on a closed curved surface which surrounds the breast. However, it is difficult to arrange a plurality of detecting elements 321 on the whole of a closed curved surface which surrounds the object (breast). Therefore, it is desirable to arrange a plurality of detecting elements 321 on a hemispherical surface, as in the present embodiment. Furthermore, the arrangement method thereof desirably involves a spiral-shaped arrangement as disclosed in Japanese Patent No. 4341987. The shape of the acoustic matching material container 36 according to the present embodiment is a shape that permits the abovementioned arrangement of detecting elements. Here, a bowl-shaped member is shown as a representative example of a container-shaped member, but the shape in not limited to this in actual practice.

Furthermore, the acoustic matching material container 36 has a shape that allows acoustic matching liquid 35 to be filled in between the holding member 11 and the detection unit 32, and also enables relative scanning with respect to the holding member 11. Therefore, the acoustic matching material container 36 has a shape composed by a hemispherical portion in which the detection unit is arranged, and a portion extending from this in the outer circumferential direction, as shown in FIG. 2. The acoustic matching material container 36 may be made from one component, or may be made by combining a plurality of components.

Here, a hemispherical member is used, but the shape of the supporter of the detecting elements is not limited to this . For example, it is also possible to use a spherical cap shape, spherical band shape, or a shape cut from an elliptical shape, or a combination of a plurality of curved surfaces or flat surfaces. Provided that the container-shaped member is capable of holding a liquid, it is possible to use this member as a support for the detecting elements. The shape of the acoustic matching material container that constitutes the receiver may also be called a bowl shape or a cup shape.

Desirably, it is also possible to arrange a light irradiation unit 31 in the acoustic matching material container 36. Thereby, since the relationship between the detection position of the photoacoustic waves and the irradiation position of the light is kept uniform, then photoacoustic wave information of more uniform quality can be acquired. The irradiation surface area of the breast onto which light can be emitted is restricted by the standards of the American National Standards Institute (ANSI). Therefore, although it is desirable to increase the irradiation intensity and the irradiation surface area in order to increase the amount of light propagated inside the breast, the irradiation surface area is restricted from the perspective of the cost of the light source, etc. Furthermore, if light is emitted onto a region where the detection sensitivity is low due to the directionality of the detecting elements 321, then the use efficiency of the amount of light is low. Therefore, it is not efficient to emit light onto the whole breast. In other words, good efficiency is achieved if the light is emitted only onto the region where the detection unit 32 which is constituted by a plurality of detecting elements 321 has high sensitivity, and therefore it is desirable to move the light irradiation unit 31, as well as the detection unit 32.

(Signal Processing and Information Processing System)

The controller 2 controls the intensity of the emitted light, the timing of light irradiation and acoustic wave reception, the movement of the relative positions of the detection unit and the object, and so on. The controller is also configured so as to be able to control the whole object information acquiring apparatus. For example, the controller may also control UV irradiation, the operation of the liquid circulator, the signal processing apparatus, the information processing apparatus, and the like.

The signal processor 4 applies an amplification process and a digital conversion process to the analog electrical signal generated by the detecting elements.

The image generation unit 6 generates image data representing property information of the interior and/or surface of the object, using this digital electrical signal. In this, it is possible to use any image reconstruction method, such as phasing addition, back projection, or the like. The generated image data may be displayed on the display unit 7, and may be stored as data for subsequent use. A display apparatus, such as a liquid crystal display, can be used for the display unit 7. The display unit 7 does not necessarily have to be included as a constituent element of the object information acquiring apparatus, and a mode can be employed in which image data is transmitted to a display that is external to the apparatus.

The controller 2, the signal processor 4 and the image generation unit 6 may be configured as circuits having respective functions, or may be provided as functions of an information processing apparatus having a processor, such as a CPU, which operates in accordance with a program.

(Light Guide Means)

The light emitted from the light source 5 in FIG. 1 is guided onto the object while being shaped to a desired light distribution shape, by optical components which are typically lenses, mirrors, and the like. Furthermore, it is also possible to propagate the emitted light via an optical waveguide path, such an optical fiber, or a optical fiber bundle, or an articulating arm incorporating mirrors, or the like, in a mirror tube, etc. The light guide means 51 includes these optical components and/or the optical waveguide path. The optical components forming the light guide means 51 are, for example, a mirror which reflects light, a lens which condenses or enlarges or changes the shape of the light, a diffusion plate which diffuses the light, and so on. Any optical components of this kind may be used, provided that the light from the light source is emitted in a desired shape onto the breast. Here, it is necessary to emit light within a range that does not exceed the maximum permitted exposure (MPE) for the segment receiving light irradiation. If it is possible to emit light of desired pulses directly onto the breast from the light source 5, and the light source 5 can be scanned together with the acoustic matching material container 36, then the photoacoustic apparatus does not need to be provided with light guide means 51.

(Liquid Circulator)

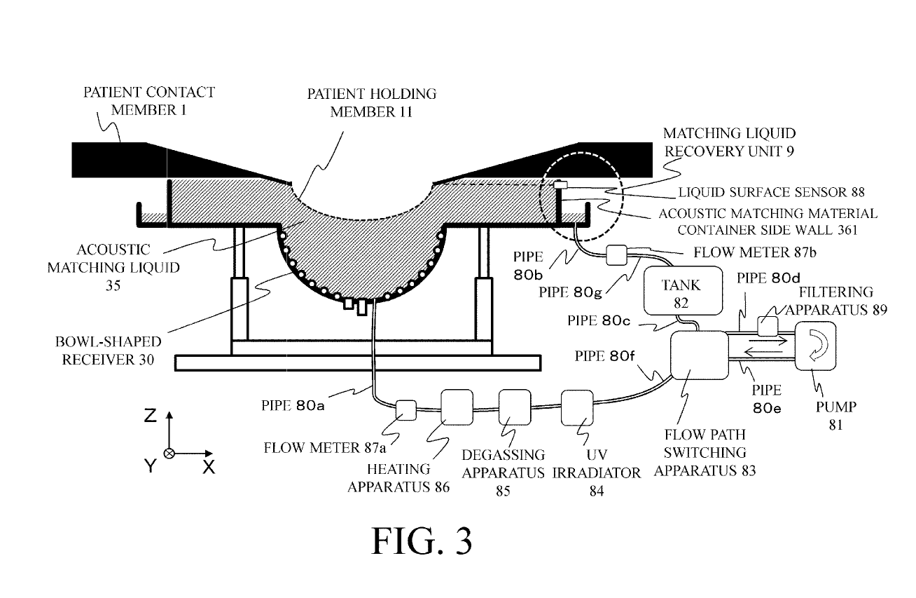

FIG. 3 is a diagram showing an example of the configuration of a liquid circulator 8 according to the present invention. The liquid circulator 8 is provided with: a pump 81, a tank 82, a flow path switching apparatus 83, a UV irradiator 84, a degassing apparatus 85, a heating apparatus 86, flow meters 87a, 87b, pipes 80a, 80b, 80c, 80d, 80e, 80f, 80g, a liquid surface sensor 88, and a filtering apparatus 89.

One example of the configuration of the liquid circulator 8 is described here. The acoustic matching liquid 35 is stored in a tank 82. When the flow path switching apparatus 83 performs a prescribed operation, the pipe 80c and the pipe 80d are connected, and the pipe 80e and the pipe 80f are connected. By operating the pump 81 in this state, the acoustic matching liquid 35 held in the tank 82 passes along the pipe 80c and the pipe 80d, through the filtering apparatus 89 and the pump 81, through the pipe 80e and the pipe 80f, and is sent to the UV irradiator 84 for sterilization. The acoustic matching liquid 35 is also processed by a defoaming (degassing) apparatus 85 which removes air and/or bubbles contained in the liquid, and a heating apparatus 86 which heats the acoustic matching liquid 35. The acoustic matching liquid 35 passes through the pipe 80a, and is supplied to the interior of the container via an inlet port which is provided at the junction between the pipe 80a and the bowl-shaped receiver 30.

The amount and flow rate of the acoustic matching liquid 35 supplied to the bowl-shaped receiver 30 is measured and controlled by the flow meter 87a. The liquid surface sensor 88 monitors whether or not the acoustic matching liquid 35 supplied to the bowl-shaped receiver 30 is filled to a sufficient level at all times. Desirably, the liquid surface sensor 88 issues a measurement error and/or warning to the display unit 7, when the liquid level has fallen below a prescribed set value.

When the acoustic matching liquid 35 continues to be supplied from the pipe 80a, the liquid overflows out from between the side wall 361 of the acoustic matching material container, and the object contact member 1, travels over the outer surface of the side wall 361 of the acoustic matching material container, and flows into a matching liquid recovery unit 9 which is configured on the outer circumference. The acoustic matching liquid 35 flows out to the pipe 80b from a discharge port provided at the junction between the matching liquid recovery unit 9 and the pipe 80b. The amount of the acoustic matching liquid 35 flowing out from the pipe 80b is measured by the flow meter 87b. The acoustic matching liquid 35 is returned again to the tank 82 via the pipe 80g.

The amount of the acoustic matching liquid entering and exiting the bowl-shape receiver 30 is monitored by the flow meter 87a and the flow meter 87b, and therefore, increase or decrease in the acoustic matching liquid 35 inside the bowl-shaped receiver 30 can be measured. Since the acoustic matching liquid 35 which has been heated and warmed seeks to rise up by convection inside the bowl-shaped receiver 30, then in order to reduce temperature irregularities, it is desirable to supply the liquid from the bottom of the bowl-shaped receiver 30 and discharge the liquid from the top thereof. Therefore, the supply port of the acoustic matching liquid 35 according to the present invention is formed in the base of the bowl-shaped receiver 30, and the liquid is discharged by recovering matching liquid 35 that has overflowed from the top of the bowl-shaped receiver, in the matching liquid recovery unit 9.

The flow path switching apparatus 83 is a mechanism which selectively opens and closes the flow path of the liquid, between a pipe which suctions acoustic matching liquid 35 by a pump, and a pipe which supplies acoustic matching liquid 35 that has passed through the pump. The flow path switching apparatus 83 can be configured by a combination of electromagnetic valves, or the like. For example, when all of the acoustic matching liquid 35 in the bowl-shaped receiver 30 is to be returned to the tank 82, then the pipe 80a, which has been used for supplying the acoustic matching liquid 35, must be used for discharging the liquid. By connecting the pipe 80f and the pipe 80d, and the pipe 80e and the pipe 80c, by means of the flow path switching apparatus 83, it is possible to return the acoustic matching liquid 35 in the bowl-shape receiver 30, to the tank 82, without operating the pump 81 in reverse.

Any type of pump may be used, provided that the pump is capable of causing a flow of the acoustic matching liquid 35. For example, in the case of a gear pump or a tube pump, or the like, it is possible to cause a flow of acoustic matching liquid 35 in both the supply and discharge directions, by reverse operation of the motor, and therefore the number of switching operations of the flow path switching apparatus 83 can be reduced.

Furthermore, the object information acquiring apparatus may also be provided with a plurality of pumps. Consequently, if the acoustic matching liquid 35 does not return to the tank 82 from the pipe 80b by the force of gravity alone, for example, then discharge from the pipe 80b and the supply of acoustic matching liquid 35 to the bowl-shaped receiver 30 which is carried out via the pipe 80a, can be performed simultaneously.

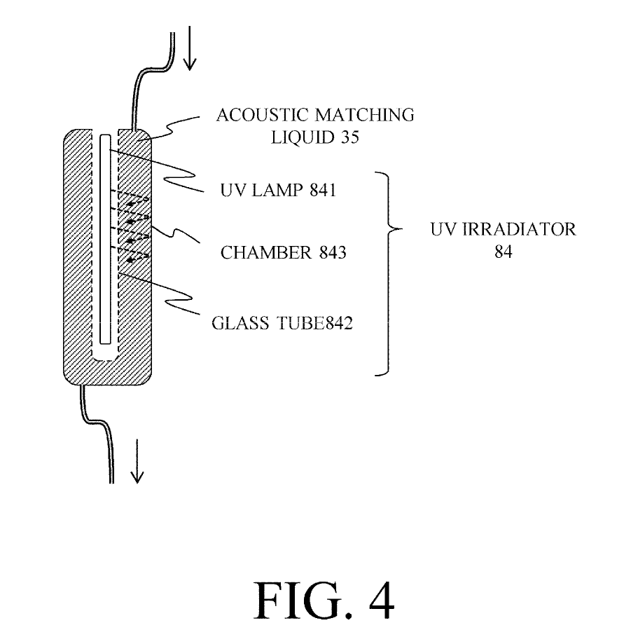

The UV irradiator 84 in the first embodiment emits UV light generated by a UV lamp 841 onto the acoustic matching liquid 35 flowing in a flow path sandwiched by a glass tube 842, as shown in FIG. 4. Consequently, the acoustic matching liquid 35 can be sterilized, and therefore decomposition of the acoustic matching liquid 35, and the growth of organic materials, such as mold, fungi, lichen etc., can be prevented. The UV light (ultraviolet light) used for sterilization is near-ultraviolet light having a wavelength of 200 to 280 nm (called “UV-C”), and, in particular, it is known that the strongest sterilizing action is obtained in the vicinity of a wavelength of 253.7 nm. Furthermore, sterilizing lamps in which the majority of the emitted light has a wavelength of 253.7 nm have been developed and are commercially available. This wavelength does not match that of the light source used in the object information acquiring apparatus of the present invention (no less than 500 nm and no more than 1200 nm), and therefore it is necessary to provide the light source for sterilization separately from the light source for measurement.

The flow path may be of any shape, provided that the arrangement enables irradiation of UV light. For example, a flow path may be configured so as to surround the UV lamp, or may be covered by a chamber 843 which reflects the UV light. Consequently, it is possible to emit light from the UV lamp 841, efficiently onto the acoustic matching liquid 35, and since the UV light reflected by the chamber 843 is emitted again onto the acoustic matching liquid 35, then the sterilizing effect is improved. Furthermore, all of the acoustic matching liquid 35 sent to the bowl-shaped receiver 30 via the pipe 80a in FIG. 3 passes along the flow path in the UV irradiator 84, and therefore, UV light can be emitted reliably onto all of the acoustic matching liquid 35 that is supplied to the bowl-shaped receiver 30. Furthermore, since the UV light only needs to be emitted while the pump is operating and the acoustic matching liquid 35 is flowing along the flow path, then wear of the UV lamp 841 is prevented and reduction in the replacement frequency and reduction in costs can be achieved.

When bubbles occur in the acoustic matching liquid 35, the propagation of the acoustic wave is shielded, thus affecting reception. Therefore, gas bubbles and dissolved gas components are removed from the acoustic matching liquid 35 by the degassing apparatus 85 shown in FIG. 3.

The degassing apparatus 85 is connected to the heating apparatus 86, and sends defoamed (and degassed) acoustic matching liquid 35 to the heating apparatus 86. Since discomfort is caused to the patient if the acoustic matching liquid 35 is excessively cold, the acoustic matching liquid 35 is heated to around body temperature by the heating apparatus 86. Furthermore, when the temperature of the acoustic matching liquid 35 changes, the speed of sound varies, which affects the received sound wave, and therefore the liquid is kept at a uniform temperature at all times.

The tank 82 has a capacity that enables storage of all of the acoustic matching liquid 35 flowing in the liquid circulator 8, including that in the bowl-shaped receiver 30, and the tank 82 is provided with a liquid inlet port and a liquid outlet port, which are not illustrated.

The connection sequence of the pump 81, the tank 82, the flow path switching apparatus 83, the UV irradiator 84, the degassing apparatus 85 and the heating apparatus 86 may be changed and optimized according to requirements.

The object information acquiring apparatus according to the present embodiment filters out dust by circulating the acoustic matching liquid, as well as emitting UV (ultraviolet light) thereon, and therefore the acoustic matching liquid is sterilized and the occurrence of organic material is suppressed. Consequently, it is possible to save the work of cleaning away dirt due to organic material in the circulation flow path of the acoustic matching liquid, and hygiene is maintained. Furthermore, since soiling by organic material is suppressed in the acoustic matching liquid, the acoustic array detector, and the object holding member, then it is possible to reduce deterioration of image quality, such as noise caused by soiling. Furthermore, the replacement frequency of the acoustic matching liquid is reduced, and the operational costs of the apparatus can be reduced.

### Second Embodiment

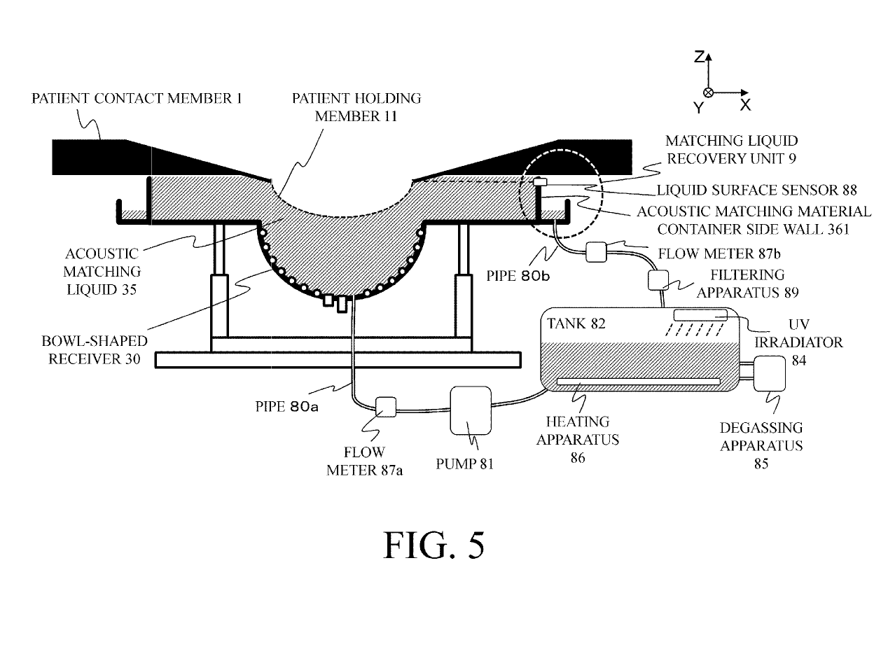

In the liquid circulator according to the first embodiment, the UV irradiator 84, the degassing apparatus 85 and the heating apparatus 86 are arranged between the tank 82 and the bowl-shaped receiver 30. In the second embodiment, the functions of the UV irradiator 84, the degassing apparatus 85 and the heating apparatus 86 are provided in the tank 82, as shown in FIG. 5.

The heating apparatus 86 is built into the tank 82, and the temperature of the acoustic matching liquid 35 is controlled to around body temperature. Furthermore, since the UV irradiator 84 is also disposed inside the tank 82, then a sterilizing effect is also obtained in the acoustic matching liquid 35 inside the tank 82. If the inner walls of the tank 82 are surface treated so as to reflect UV light, then the sterilizing effect is increased by reflection of the UV light. The degassing apparatus 85 may be built into the tank 82, or an external degassing apparatus may be connected to the tank 82 and degassing carried out while circulating liquid between the tank 82 and the degassing apparatus 85.

The acoustic matching liquid 35 which has been sterilized, degassed and heated inside the tank 82 is supplied by the pump 81 to the bowl-shaped receiver 30 via the pipe 80a. The acoustic matching liquid 35 supplied to the bowl-shaped receiver 30 flows out from the pipe 80b via the matching liquid recovery unit 9, similarly to the first embodiment, and is recovered again in the tank 82, where the liquid is sterilized, degassed and heated.

In the configuration according to the first embodiment, UV irradiation, degassing and heating are only possible while the acoustic matching liquid 35 is passing along the flow path, but with the configuration according to the second embodiment, UV irradiation, degassing and heating of the acoustic matching liquid 35 are also possible while the acoustic matching liquid 35 is being held in the tank. Therefore, by using the acoustic matching liquid 35 so that the liquid is held in the tank 82 for the time required for UV irradiation, degassing and heating, a sufficient effect is obtained even when using a UV irradiator 84, degassing apparatus 85 and heating apparatus 86 which have a low processing capacity per unit time.

Furthermore, with the configuration according to the second embodiment, it is possible to simplify the flow path switching apparatus 83. Moreover, by using a pump 81 in which the direction of the conveyed acoustic matching liquid 35 is reversible, it is also possible to eliminate the flow path switching apparatus 83.

### Third Embodiment

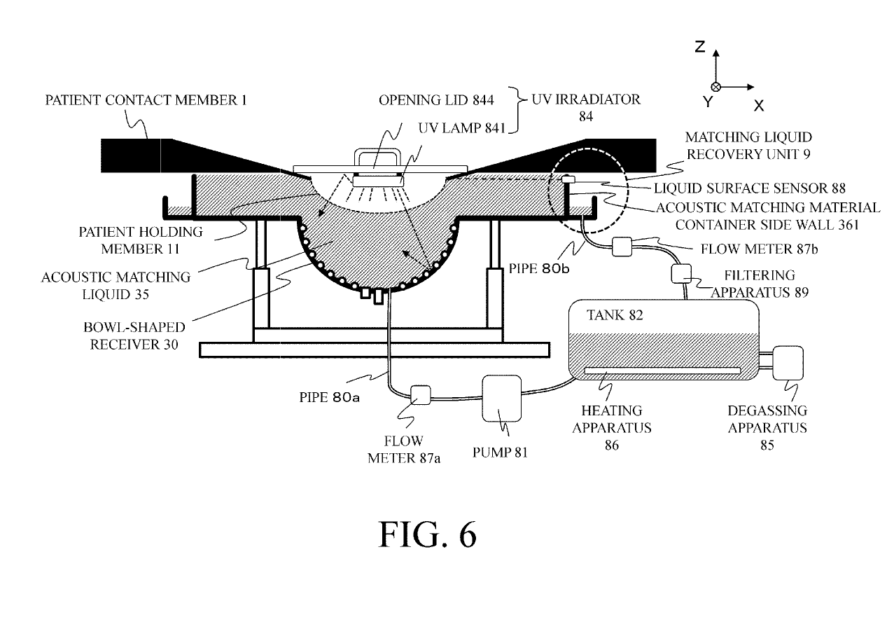

In the first embodiment, a configuration is described in which the UV irradiator 84 is disposed in the flow path and in the second embodiment, a configuration is described in which the UV irradiator 84 is disposed inside the tank 82. In the third embodiment, as shown in FIG. 6, the UV irradiator 84 is disposed above the holding member 11. In these cases, the ultraviolet light is emitted onto the acoustic matching liquid 35 via the holding member.

The UV irradiator 84 may be disposed directly on top of the holding member 11.

Furthermore, as shown in FIG. 6, a lid-shaped member, such as the opening lid 844, which is capable of shutting off the breast insertion opening 13 may also be provided. By installing the UV lamp 841 on the opening lid 844, damaging of the surface of the holding member 11 is prevented. Furthermore, it is desirable if the opening lid 844 has light shielding properties, since the emitted ultraviolet light does not leak outside the apparatus, which enables protection of the user's eyes, etc.

According to the present embodiment, a sterilizing effect is obtained by emitting UV light onto the holding member 11 which comes into direct contact with the patient, in addition to the acoustic matching liquid, and therefore hygiene is maintained. On the other hand, since the UV irradiator 84 is placed on top of the breast introduction opening, then it is not possible to use the UV irradiator 84 while the patient's breast is inserted and is being measured. Therefore, irradiation of UV light should be carried out by circulating the acoustic matching liquid 35 inside the bowl-shaped receiver 30 while the acoustic matching liquid 35 is being degassed and heated prior to starting measurement.

In FIG. 6, the sterilizing effect is raised further by reflecting the UV light through providing a surface treatment for reflecting the UV light on the installation surface of the UV lamp 841 on the opening lid 844, and on the surfaces of the bowl-shaped receiver on the sides that contact the acoustic matching liquid 35.

The liquid circulation method may use a configuration in which the liquid is circulated by providing the degassing apparatus 85 and the heating apparatus 86 outside the tank 82, as in the first embodiment, or a configuration in which the liquid is circulated by installing these apparatuses in the tank 82, as in the second embodiment.

### Fourth Embodiment

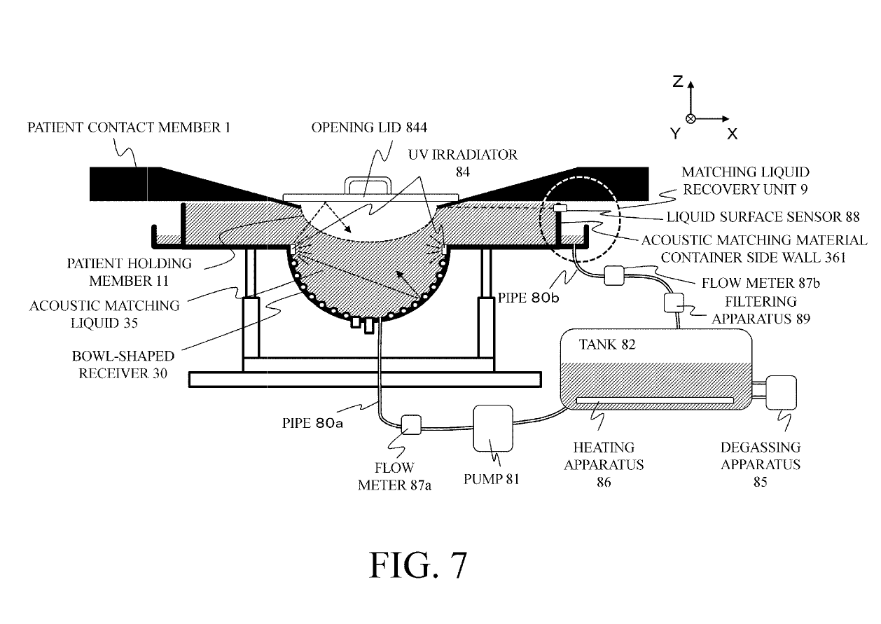

In the third embodiment, a configuration was described in which the UV irradiator is disposed on top of the holding member 11. In the fourth embodiment, as shown in FIG. 7, the UV irradiator 84 is disposed in the bowl-shaped receiver 30.

The installation position of the UV irradiator 84 may be set to a position within the bowl-shaped receiver 30 where the apparatus can irradiate UV light directly onto the acoustic matching liquid 35, and which does not obstruct the scanning action or the acoustic waves from the object. For example, by disposing the UV irradiator 84 on the circumferential edge of the upper end portion of the spherical shape of the bowl-shaped receiver 30, in addition to the sterilizing effect of the acoustic matching liquid 35, it is also possible to achieve a sterilizing effect of the holding member 11, and an effect in suppressing mold and lichen in the detection unit provided inside the bowl-shaped receiver 30.

The circulation method of the acoustic matching liquid 35 is similar to that of the third embodiment. Desirably, the opening lid 844 is provided, and UV light is emitted while the liquid is circulated inside the bowl-shaped receiver 30, during the degassing and heating of the acoustic matching liquid 35 prior to the start of measurement . In order to eliminate the risk of damage to the eyes due to directly looking at the ultraviolet light emitted from the UV irradiator 84, or burning due to irradiation of light onto the skin, it is desirable for the apparatus to be provided with an interlock (not illustrated) whereby UV light cannot be emitted while the opening lid is open.

Furthermore, similarly to the third embodiment, the sterilizing effect is raised further by reflecting the UV light through providing a surface treatment for reflecting the UV light on the installation surface of the UV lamp 841 on the opening lid 844, and on the surfaces of the bowl-shaped receiver on the sides that contact the acoustic matching liquid 35.

The object information acquiring apparatus according to the present invention filters out dust by circulating the acoustic matching liquid, as well as irradiating UV (ultraviolet light) thereon, and therefore the acoustic matching liquid is sterilized and the occurrence of organic material is suppressed. In this way, according to the present invention, it is possible to suppress deterioration of the liquid used as an acoustic matching agent. As a result of this, the work of cleaning away soiling caused by organic material in the circulation flow path of the acoustic matching liquid can be saved, and a hygienic environment as required in a diagnostic apparatus can be maintained. Furthermore, since soiling by organic material is suppressed in the acoustic matching liquid, the acoustic array detector, and the object holding member, then it is possible to suppress deterioration of image quality, such as noise caused by soiling. Furthermore, the replacement frequency of the acoustic matching liquid is reduced, and the operational costs of the apparatus can be reduced.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment (s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. Provisional Application No. 62/046,352, filed on Sep. 5, 2014, which is hereby incorporated by reference herein in its entirety.

What is claimed is:

1. An object information acquiring apparatus, comprising:
a receiver provided with a plurality of detecting elements which receive an acoustic wave from an object and generate an electrical signal, and provided with a container-shaped supporter which supports the plurality of detecting elements;
a tank which holds an acoustic matching liquid;
a liquid circulator which is provided with a flow path connecting the tank with a liquid inlet port and a liquid discharge port provided in the receiver and which controls a flow of the acoustic matching liquid;
a UV irradiator which irradiates the acoustic matching liquid with ultraviolet light; and
a processor which uses the electrical signal to generate property information on the interior of the object.

2. The object information acquiring apparatus according to claim 1, wherein the UV irradiator irradiates the acoustic matching liquid in the flow path with ultraviolet light.

3. The object information acquiring apparatus according to claim 1, wherein the UV irradiator is provided inside the tank.

4. The object information acquiring apparatus according to claim 1, further comprising a holding member which is disposed above the receiver and which holds the object, wherein
the UV irradiator irradiates the acoustic matching liquid via the holding member.

5. The object information acquiring apparatus according to claim 4, further comprising an object contact member which supports a patient in a prone lying posture, wherein
the object is a breast of the patient inserted via an opening in the object contact member.

6. The object information acquiring apparatus according to claim 5, further comprising a lid-shaped member which closes off the opening, wherein
the UV irradiator is installed on the lid-shaped member.

7. The object information acquiring apparatus according to claim 1, wherein the UV irradiator is provided on the supporter of the receiver.

8. The object information acquiring apparatus according to claim 1, wherein the liquid inlet port is provided in a lower portion of the supporter of the receiver.

9. The object information acquiring apparatus according to claim 1, wherein the liquid discharge port is provided in an upper portion of the supporter of the receiver.

10. The object information acquiring apparatus according to claim 1, further comprising a light source, wherein
the acoustic wave is emitted from the object which has been irradiated with light from the light source.
